(19) United States
(12) Patent Application Publication
(10) Pub. No.: US 2021/0283176 A1
(43) Pub. Date: Sep. 16, 2021

(54) CALCIUM BINDER FOR USE IN PREVENTING STILLBIRTH

(71) Applicant: Nutreco IP Assets B.V, Boxmeer (NL)

(72) Inventor: Pieter Langendijk, Amersfoort (NL)

(73) Assignee: Nutreco IP Assets B.V, Boxmeer (NL)

(21) Appl. No.: 16/607,997

(22) PCT Filed: Apr. 24, 2018
(86) PCT No.: PCT/NL2018/050260

§ 371 Date: Oct. 24, 2019

(30) Foreign Application Priority Data
Apr. 25, 2017 (NL) 2018777

(51) Int. Cl. A61K 33/42; A23K 50/30; A23K 20/26

(52) U.S. Cl. CPC A61K 33/42; A23K 20/26; A23K 50/30

(57) ABSTRACT

This disclosure pertains to a calcium binder for use in improving perinatal survival in the offspring of a sow, and in reducing or preventing stillbirth of piglets. This disclosure also pertains to compositions comprising a calcium binder, particularly in combination with a chloride salt to supplement a diet of an animal, in particular, a pregnant animal.

### CROSS-REFERENCE TO RELATED APPLICATIONS

This application is a national phase entry under 35 U.S.C. § 371 of International Patent Application PCT/NL2018/050260, filed Apr. 24, 2018, designating the United States of America and published as International Patent Publication WO 2018/199748 A1 on Nov. 1, 2018, which claims the benefit under Article 8 of the Patent Cooperation Treaty to Dutch Patent Application Serial No. 2018777, filed Apr. 25, 2017.

### TECHNICAL FIELD

This disclosure, in general, pertains to the field of maximizing the reproductive performance of animals, in particular, mammals, specifically a swine. This disclosure, in particular, pertains to maximizing the performance of animals and their offspring by adapting their nutrition.

### BACKGROUND

Maximizing the performance of pregnant animals and their offspring has for a long time been a major objective of nutritionists. Inter alia feed-processing technologies, amino acid supplementation and increased dietary energy density have been used to address the objective of maximizing this performance.

One important aspect of maximizing reproductive performance is optimizing the health of the pregnant animal and its (unborn) offspring during parturition. In particular, it is believed that this may reduce perinatal mortality of the offspring, such as stillbirth (intrapartum mortality) and neonatal mortality. In particular, stillbirth in piglets is mainly caused by oxygen insufficiency, due to repeated episodes of reduced blood perfusion of the placenta, caused by uterine contractions compressing the placenta, and due to stretch and sometimes rupture of the umbilical cord as the foetus travels through the uterus and the birth canal. The oxygen insufficiency leads to increasing blood lactate levels, a decreasing blood pH, and a reduction in extra-cellular fluid base excess in the foetus, a condition referred to as acidosis. When this condition aggravates, this can result in piglet death. Short term oxygen insufficiency is reversible; however, prolonged oxygen insufficiency may lead to irreversible effects and finally, death, and hence it is understandable that stillbirth occurs mainly in piglets born at the end of the birth order. For the same reasons, stillbirth rate is higher in sows that take long to farrow and therefore, the effect of birth order is aggravated in sows with prolonged parturition. It is commonly believed that one of the main causes for prolonged parturition is a reduced concentration of freely available calcium (ionic Calcium, or “iCa”) in maternal circulation. Calcium is involved in regulating the force and coordination of uterine contractions, and insufficient iCa in the blood results in poor muscle function, and as a consequence, prolonged parturition. For this reason, calcium supplementation to the diet of pregnant animals is often proposed as a measure to reduce perinatal mortality.

However, the influence of nutrition on maximizing performance, in particular, of reproductive performance, has not been conclusively explored yet.

### BRIEF SUMMARY

Described are means and methods to increase reproductive performance of a pregnant animal, in particular, to decrease perinatal mortality of offspring, reduce the number of stillbirths, and thereby improve perinatal survival.

Completely contrary to the prior art teaching that it is advantageous to supplement the diet with extra calcium, it was found that the addition of a calcium binder to the diet has a positive effect on perinatal mortality, in particular, on a reduction of stillbirth. Without being bound to theory, it is believed that by effectively lowering the amount of available calcium in the animal's diet, the animal's natural capability to release calcium from its mineral reserves (in particular, from the bone) might be stimulated.

Thus, it has surprisingly been found that it is advantageous to use a calcium binder such as phosphoric acid for oral administration to improve perinatal survival and to prevent stillbirths in sows. In particular, it has been found that the number of stillborn offspring of the pregnant animal and/or the percentage of animals having one or more stillborn offspring can be reduced in this way.

### Definitions

An animal's diet is the habitual nourishment of the animal, including feed (solid and liquid feed) and drinking water.

A calcium binder is a compound that prevents calcium ions from being dissolved in water in a concentration of more than 10 mM, in particular, more than 1 mM.

A salt is insoluble in water if an aqueous solution of the salt contains less than 1 mmol of the salt per liter of water at room temperature.

A dietary supplement is a product intended for ingestion, which contains a dietary ingredient intended to add nutritional value to the diet. The product can, for example, be added to the solid feed of the animals (in which case the supplement is often referred to as a top-dress) or to the drinking water (in which case the supplement is often referred to as a drinking water additive).

Perinatal mortality is the total of stillbirths and neonatal deaths.

Stillbirth is the phenomenon where offspring is born dead, but has died only shortly before (in particular, up to 24-48 hours) or during parturition.

Neonatal death is the phenomenon where offspring dies shortly after birth, typically within 10 days after birth.

### DETAILED DESCRIPTION

Described is a calcium binder, such as phosphoric acid, to improve perinatal survival in the offspring of an animal.

The animal is preferably a pregnant animal, more preferably a pregnant farming animal, even more preferably a pregnant monogastric farming animal, and most preferably a pregnant sow. As such, the offspring is preferably piglets.

In an embodiment the calcium binder, such as phosphoric acid, is added to the animal's diet (which also encompasses enriching the diet in certain components by extracting other ones). This was found to be a convenient way to provide for oral administration. The calcium binder, such as phosphoric acid, may be added to the drinking water or feed of the animal. For example, the calcium binder may be contained in a composition, which is in the form of a premix, a liquid, a powder, granules, a pellet, a dragee, a tablet, a pill, or a capsule.

In particular, the calcium binder, such as phosphoric acid, may be added to the drinking water of the animal. The advantage thereof is that pregnant animals, even right before parturition, normally show regular or even increased drinking behaviour, whereas they might decrease or even cease the eating of solid feed. Thus, consumption of the calcium binder, such as phosphoric acid, may be continued particularly at the point in time when approaching parturition, which is when the iCa in sows is at its highest, and when the calcium binder is most effective.

In yet another embodiment the calcium binder, such as phosphoric acid, is administered in a period of 0 to 5 days before parturition, in particular, at least in a period of 1 to 5 days before parturition (which does not exclude that the calcium binder is also administered earlier or later, for example, during parturition).

In a particular embodiment, the calcium binder is an anion that forms a water insoluble salt with calcium ions in aqueous solution. Such an ion may, for example, be a conjugated base ion.

Suitable calcium binders include, without limitation, any compounds that are capable of binding free calcium the ruminant gastro-intestinal tract whereby the free calcium cannot be absorbed by the animal. Thus, the natural calcium regulating defence mechanism of the animal is triggered. Such compounds include, without limitation, phosphoric acid, oxalic acid, sodium oxalate, phytic acid, a phytate, a clay mineral including zeolite, sodium diethylene acetic acid, ethylene diaminetetraacetic acid (EDTA), the sodium salts of EDTA Na₂EDTA and Na₄EDTA, trisodium nitrilotriacetate monohydrate, trisodium ntrioacetate, pentasodium diethylenetriaminepentaacetate, tri sodium N-hydroxyethylethylenediaminetriacetate, citric acid, a citrate, a polyphosphate, a tripolyphosphate, a phosphate, a cellulose phosphate, glutamic acid N,N-diacetic acid (GLDA), a sodium salt of GLDA, a potassium salt of GLDA, methylglycine-N,N-diacetic acid (MGDA), a sodium salt of MGDA, a potassium salt of MGDA, ethylenediamine N,N′-disuccinic acid (EDDS), a sodium salt of EDDS, a potassium salt of EDDS, iminodisuccinic acid (IDS), a sodium salt of IDS, or a potassium salt of IDS, or a calcium-free derivative of any such compounds.

The calcium binder may advantageously be phosphoric acid or a phosphate, particularly when the calcium binder is added to drinking water. Phosphoric acid has the interesting property that it does not form a water insoluble salt with calcium ions at low pH (i.e., when in solution prior to administration to an animal, it remains in soluble form, even when formulated with a calcium salt such as calcium chloride), whilst it precipitates with calcium ions at physiological pH (i.e., in the GI tract of the animal). The calcium binder is preferably an anion that forms a water insoluble salt with calcium ions at physiological small intestinal pH (e.g., a pH in the range of 6-7.5, preferably 6.5-7.5), but not at acidic pH (e.g., a pH below 6, preferably below 5.5).

The calcium binder may be encapsulated by any appropriate encapsulating material. A compound suitable for encapsulation of calcium binder is a compound selected from the group consisting of a fat, a non-calcium derivative of a fat such as a soap and a stearate, a protein, a polysaccharide, a cellulose and a derivative of any such compound, a gum, a glycol and gelatine.

The addition of a calcium binder to an animal's diet is particularly advantageous when combined with administration of a chloride salt, particularly calcium chloride.

This disclosure will now be further explained using the following non-limiting example.

### EXAMPLES

### Example 1

A calcium binder (phosphoric acid, PhA)) was added in a concentration of 0.01 mol/liter (corresponding to 1 g/L of phosphoric acid). The diets fed to the sows were the same. The results of the study are indicated here below in table 1.

TABLE 1
Effect of phosphoric
acid on stillbirth

| | PhA* | Control |
|---|---|---|
| Number of sows | 25 | 24 |
| Stillborn piglets, # | 1.1 | 1.6 |

*PhA = phosphoric acid

In this experiment, stillbirth was reduced from 1.6 to 1.1 piglets per litter. It is believed that binding of dietary calcium in the gut and the inherent effect of reducing the amount of calcium available to the animal prior to parturition, has as a consequence that the animal is triggered to mobilise calcium from its own mineral reserves in the bones. Therefore, when demand for calcium is increased as is the case in parturition, the animal is more prepared to face this demand, and better equipped to maintain blood Ca levels. In the example described above, blood calcium levels were indeed increased by the treatments provided (data not shown).

1.-10. (canceled)

11. A method of improving perinatal survival of the offspring of an animal, the method comprising:
administering a calcium binder to the animal
so as to improve perinatal survival of the animal's offspring.

12. The method according to claim 11, wherein the calcium binder is added to the diet of a pregnant animal.

13. The method according to claim 12, wherein the calcium binder is added to the drinking water or feed of the pregnant animal.

14. The method according to claim 12, wherein the calcium binder is administered in a period of 0 to 5 days before parturition.

15. The method according to claim 14, wherein the calcium binder is administered in a period of 1 to 5 days before parturition.

16. The method according to claim 11, wherein the calcium binder is an anion that forms a water insoluble salt with calcium ions at a pH in the range of 6-7.5, but not at a pH below 6.

17. The method according to claim 16, wherein the calcium binder is phosphoric acid.

18. The method according to claim 17, further comprising:
administering a chloride salt to the animal.

19. The method according to claim 13, which is administered in the drinking water of the pregnant animal.

20. The method according to claim 19, further comprising:
administering a chloride salt to the animal.

21. The method according to claim 11, wherein the animal is a sow.

22. A method of reducing or preventing stillbirth of a neonate of an animal, the method comprising:
administering a calcium binder to the animal
so as to reduce or prevent stillbirth of neonates.

23. The method according to claim 22, wherein the calcium binder is added to the diet of a pregnant animal.

24. The method according to claim 23, wherein the calcium binder is added to the drinking water or feed of the pregnant animal.

25. The method according to claim 23, wherein the calcium binder is administered in a period of 0 to 5 days before parturition.

26. The method according to claim 25, wherein the calcium binder is administered in a period of 1 to 5 days before parturition.

27. The method according to claim 22, wherein the calcium binder is an anion that forms a water insoluble salt with calcium ions at a pH in the range of 6-7.5, but not at a pH below 6.

28. The method according to claim 27, wherein the calcium binder is phosphoric acid.

29. The method according to claim 28, further comprising:
administering a chloride salt to the animal.

30. The method according to claim 24, which is administered in the drinking water of the pregnant animal.

31. The method according to claim 30, further comprising:
administering a chloride salt to the animal.

32. The method according to claim 22, wherein the animal is a sow.
